(19) United States
(12) Patent Application Publication
(10) Pub. No.: US 2008/0194911 A1
(43) Pub. Date: Aug. 14, 2008

(54) Camera Imaging Apparatus With an Endoscope

(75) Inventor: Sung Yong Lee, Gangwon-do (KR)

Correspondence Address: OSHA LIANG L.L.P., 1221 MCKINNEY STREET, SUITE 2800, HOUSTON, TX, 77010, US

(73) Assignee: CARETEK CO., LTD., Gangwon-Do (KR)

(21) Appl. No.: 11/994,109

(22) PCT Filed: Jun. 26, 2006
(86) PCT No.: PCT/KR2006/002447

§ 371 Date: Apr. 21, 2008

(30) Foreign Application Priority Data
Jun. 27, 2005 (KR) 10-2005-0055807

(51) Int. Cl. A61B 1/04

(52) U.S. Cl. 600/109

(57) ABSTRACT

Disclosed is a camera imaging apparatus with an endoscope includes a refraction stacked material which is stacked to make a plenty of circle plate to be retracted, a driving wire which penetrates the circle plate of refraction stacked material, an elastic supporter which makes the circle plate of refraction stacked material to be able to be refracted, a camera which is installed on the front of the refraction stacked material, and a hose which is installed on the rear of the refraction stacked material.

### TECHNICAL FIELD

The present invention relates to a camera imaging apparatus with an endoscope capable of making a film of a scene at a narrow space such as the internal structure of the human body, the interior of a machine, and the interior of a pipe in the field of a medicine and an industry.

### BACKGROUND ART

A camera imaging apparatus with an endoscope has been utilized to make a film of a scene at a narrow space such as the internal structure of the human body, the interior of a machine, and the interior of a pipe. In the camera imaging apparatus, a camera is installed at the front of a hose capable of folding and also is organically connected to a wire of the interior of the hose.

Using the camera imaging apparatus having an endoscope, the front part of an endoscope provided with the camera in a case of making a film of the internal structure of the human body, the interior of a machine, or the interior of a pipe is inserted in a film target, and thereafter the photographing is being carried out with adjusting a photographing angle of the camera by pulling the wire.

Because a driving mechanism with the wire for adjusting a photographing angle of the camera is very accurate and complicated, it is often occurred to get out of order and it is difficult to accurately control the photographing angle of the camera. Also, it has only to allot a high price because of complication.

### DISCLOSURE OF INVENTION

### Technical Problem

To solve the above problems, it is an object of the invention to provide a camera imaging apparatus with an endoscope capable of accurately controlling a photographing angle of a camera and of having a simple structure and it is almost little occurred to make out of order, as well as having an economic manufacturing cost.

### Technical Solution

In order to accomplish the above object, the present invention provides first embodiment of a camera imaging apparatus with an endoscope includes a refraction stacked material which is stacked to make a plenty of circle plate to be refracted, a driving wire which penetrates the circle plate of refraction stacked material, an elastic supporter which makes the circle plate of refraction stacked material to be able to be refracted, a camera which is installed on the front of the refraction stacked material, and a hose which is installed on the rear of the refraction stacked material and which penetrates the above driving wire; and a refraction stacked material 10 having a plenty of circle plates 11 including a supporter 11a which is formed protuberantly less than a half circle to the upper of one side, a pair of supporting grooves 11b and 11c which is formed oppositely against across the outside of the supporter 11a, and a pair of penetration holes 11d and 11e which is formed oppositely against between the supporting grooves 11b and 11c, wherein the refraction stacked material 10 is realized in carrying out processes of making many circle plates 11 to be stacked to make the supporter 11a to be positioned at one part, and of inserting an elastic supporter 30 in the supporting grooves 11b and 11c and installing thereon, successively to make the penetration of a driving wire 20 through a pair of penetration holes 11d and 11e.

It is an object to provide second embodiment of a camera imaging apparatus with an endoscope includes a refraction stacked material which is stacked to make a plenty of circle plate to be refracted, a driving wire which penetrates the circle plate of refraction stacked material, an elastic supporter which makes the circle plate of refraction stacked material to be able to be refracted, a camera which is installed on the front of the refraction stacked material, and a hose which is installed on the rear of the refraction stacked material and which penetrates the above driving wire; the refraction stacked material 110 having a plenty of circle plates 111 including a supporter 111a which is formed protuberantly less than a half circle to the upper of one side, a pair of supporting grooves 111b and 111c which is formed oppositely against across the outside of the supporter 111a, and a pair of penetration holes 111d and 111e which is formed oppositely against between the supporting grooves 111b and 111c; and being realized in carrying out processes of making many circle plates 111 to be stacked to be positioned at one part, and of inserting an elastic supporter 130 in the supporting grooves 111b and 111c and installing thereon, successively to make the penetration of a driving wire 120 through the penetration holes 111d and 111e. At this time, the elastic supporter 130 is formed in that a concave groove 130a which is inserted in the border of the supporting grooves 111b and 111c has a constant distance so that the circle plates 111 may be keep a maintenance with a constant separation.

To accomplish the above object, It is an object to provide third embodiment of the camera imaging apparatus with an endoscope includes a refraction stacked material which is stacked to make a plenty of circle plate to be refracted, a driving wire which penetrates the circle plate of refraction stacked material, an elastic supporter which makes the circle plate of refraction stacked material to be able to be refracted, a camera which is installed on the front of the refraction stacked material, and a hose which is installed on the rear of the refraction stacked material and which penetrates the above driving wire; a refraction stacked material 210 having first to third circle plates 211, 212, and 213 has four penetration holes 211a, 212a, and 213a formed with a predetermined separation of 90° on the center; the elastic supporter 230 representing first and second elastic supporters 230a and 230b which are respectively made of a coil spring; first to third circle plates 211, 212, and 213 being respectively stacked so that a pair of first elastic supporter 230a is installed between penetration holes 211a and 212a formed oppositely against first and second circle plates 211 and 212 and a pair of second elastic supporter 230b is installed between penetration holes 212a and 213a positioned perpendicularly to first elastic supporter 230a formed between second and third circle plates 212 and 213; and the driving wire 220 penetrating corresponding penetration holes 211a, 212a, and 213a of first to third circle plates 211, 212, and 213 and first and second elastic supporter 230a and 230b.

### BRIEF DESCRIPTION OF THE DRAWINGS

FIG. 1 is a disassembled perspective view showing first embodiment of the camera imaging apparatus with an endoscope according to the present invention;

FIG. 2 is a view showing a state at which a camera is able to be moved to first direction by a refraction stacked material in the camera imaging apparatus in FIG. 1;

FIG. 3 is a disassembled perspective view showing second embodiment of a camera imaging apparatus with an endoscope according to the present invention;

FIG. 4 is a view showing a state at which a camera is able to be moved to second direction by a refraction stacked material in the camera imaging apparatus in FIG. 3;

FIG. 5 is a disassembled perspective view showing third embodiment of a camera imaging apparatus with an endoscope according to the present invention; and

FIG. 6 is a view showing a state at which a camera is able to be moved to fourth direction by a refraction stacked material in the camera imaging apparatus in FIG. 5.

### BEST MODE FOR CARRYING OUT THE INVENTION

Hereinafter, preferred embodiments of the camera imaging apparatus according to the present invention will be described in detail referring to the accompanying drawings.

A camera imaging apparatus with an endoscope includes a refraction stacked material which is refracted to a predetermined direction, a driving wire which makes the refraction stacked material refracted, an elastic supporter which is supported so that the refraction stacked material may be made to be refracted, a camera installed on the front of the refraction stacked material, and a hose which supports the rear of the refraction stacked material. Three embodiments are divided by a refraction direction of the refraction stacked material in such camera imaging apparatus with an endoscope and will be one by one explained as follows.

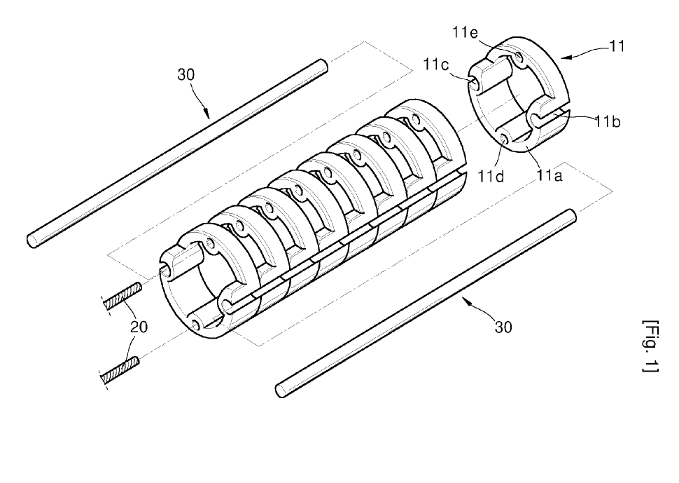

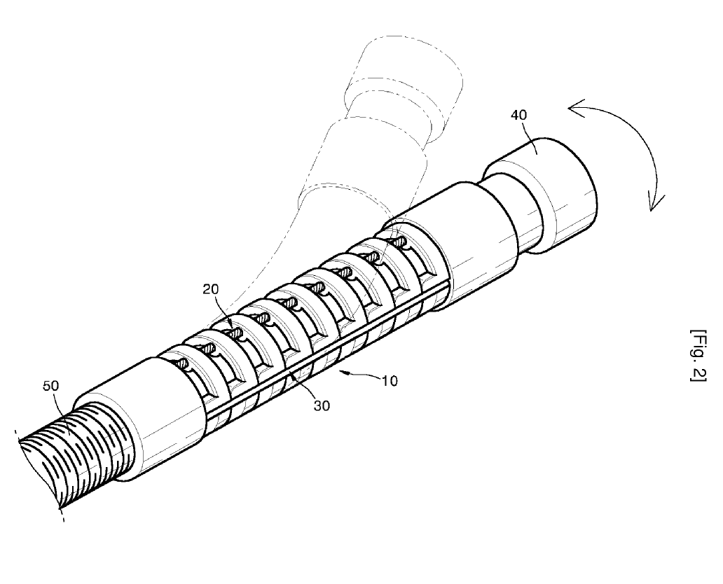

FIG. 1 is a disassembled perspective view showing first embodiment of a camera imaging apparatus with an endoscope according to the present invention, and FIG. 2 is a view showing a state at which a camera is able to be moved to first direction by a refraction stacked material in the camera imaging apparatus in FIG. 1.

As shown in FIGS. 1 and 2, the first embodiment of a camera imaging apparatus with an endoscope according to the present invention includes a refraction stacked material 10 which is stacked to make a plenty circle plate 11 to be refracted, a driving wire 20 which penetrates the refraction stacked material 10, an elastic supporter 30 which makes the refraction stacked material 10 to be able to be refracted, a camera 40 which is installed on the front of the refraction stacked material 10, and a hose 50 which is installed on the rear of the refraction stacked material 10. At this time, an adjustment part (not shown) capable of making the camera imaging apparatus to be adjusted is installed on the rear of the hose 50. Further, the camera 40 is a known digital camera to be photographed by an image sensing device in a method of a chargecoupled device.

The circle plate 11 comprised of the refraction stacked material 10 includes a supporter 11a which is formed protuberantly more than a half circle to the upper of one side, a pair of supporting grooves 11b and 11c which is formed oppositely against across the outside of the supporter 11a, and a pair of penetration holes 11d and 11e which is formed oppositely against between the supporting grooves 11b and 11c.

The refraction stacked material 10 is realized in carrying out processes of making many circle plates 11 to be stacked, and of inserting an elastic supporter 30 in the supporting grooves 11b and 11c and installing thereon, successively to make the penetration of a driving wire 20 through a pair of penetration holes 11d and 11e.

At the same time, the end part of the driving wire 20 is fixed to the upper of the refraction stacked material 10 and the lower thereof is coupled to the adjustment part via the hose 50.

The elastic supporter 30 makes the application of an elasticity force so that a plenty of stacked circle plates 11 may be formed in a straight line. Such elastic supporter 30 is made of an elastic material having an elastically big coefficient such as a spring, a urethane, and a silicone.

In such structure, the driving wire 20 which penetrate the penetration hole 11e of the circle plate 11 is pushed and pulled from the adjustment part of at the rear of the hose 50, and as shown in FIG. 2, the camera 40 installed on the front of the refraction stacked material 10 is moved to an arrow direction. And, the refraction stacked material 10 may be formed in a straight line by the elasticity of the elastic supporter 30, when the pushed or pulled driving wire 20 is released from the adjustment part, simultaneously with returning to an initial state in a camera 40.

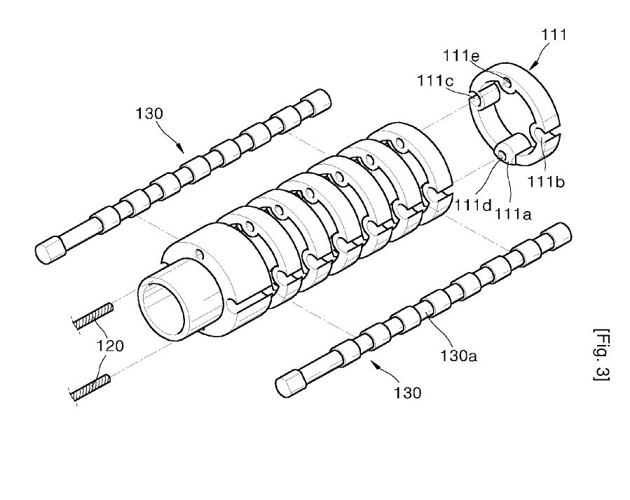

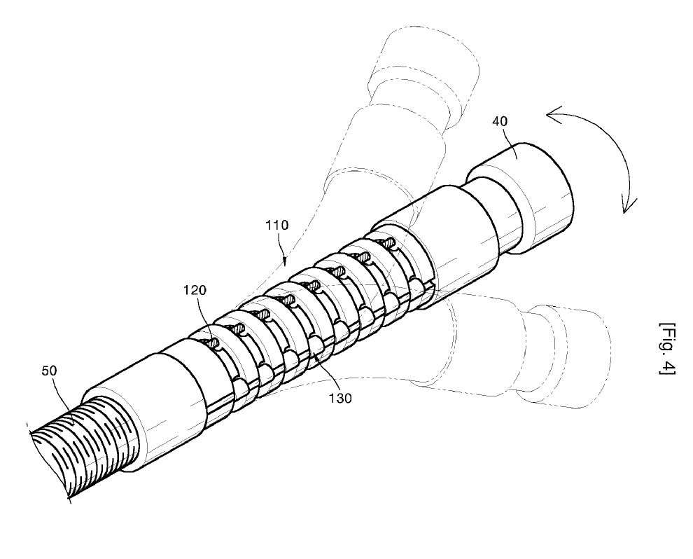

FIG. 3 is a disassembled perspective view showing second embodiment of the camera imaging apparatus with an endoscope according to the present invention, and FIG. 4 is a view showing a state at which a camera is able to be moved to second direction by a refraction stacked material in the camera imaging apparatus in FIG. 3.

As shown in FIGS. 3 and 4, second embodiment of the camera imaging apparatus with an endoscope according to the present invention includes a refraction stacked material 110 which is stacked to make a plenty of circle plates 111 to be refracted, a driving wire 120 which penetrates the refraction stacked material 110, an elastic supporter 130 which makes the refraction stacked material 110 to be able to be refracted, a camera 140 which is installed on the front of the refraction stacked material 110, and a hose 150 which is installed on the rear of the refraction stacked material 110. At this time, an adjustment part (not shown) capable of making the camera imaging apparatus to be adjusted is installed on the rear of the hose 150. Further, the camera 140 is a known digital camera to be photographed by an image sensing device in a method of a chargecoupled device.

The circle plates 111 comprised of the refraction stacked material 110 includes a supporter 111a which is formed protuberantly less than a half circle to the upper of one side, a pair of supporting grooves 111b and 111c which is formed oppositely against across the outside of the supporter 111a, and a pair of penetration holes 111d and 111e which is formed oppositely against between the supporting grooves 111b and 111c.

Herein, a different point of second embodiment from first embodiment is that the size of a supporter 111a in second embodiment is smaller than that of a supporter 11a in first embodiment and the supporting grooves 111b and 111c in second embodiment is formed oppositely against across the outside of the supporter 111a, compared to that the supporting grooves 11b and 11c in first embodiment is formed oppositely against across the inside of the supporter 11a.

The refraction stacked material 110 is realized in carrying out processes of making many circle plates 111 to be stacked to be positioned at one part, and of inserting an elastic supporter 130 in the supporting grooves 111b and 111c and installing thereon, successively to make the penetration of a driving wire 120 through the penetration holes 111d and 111e.

At the same time, the end part of the driving wire 120 is fixed to the upper of the refraction stacked material 110 and the lower thereof is coupled to the adjustment part via the hose 150.

The elastic supporter 130 makes the application of an elasticity force so that a plenty of stacked circle plates 111 may be formed in a straight line. At this time, the elastic supporter 130 is preferable that with a constant distance is formed a concave groove 130a which is inserted in the border of the supporting grooves 111b and 111c so that the circle plates 111 may be keep a maintenance with a constant separation. Such elastic supporter 130 is made of an elastic material having an elastically big coefficient such as a spring, a urethane, and a silicone.

In such structure, the driving wire 120 which penetrate the penetration hole 111e of the circle plate 111 from the adjustment part on the rear of hose or the driving wire 120 which penetrate the penetration hole 111d of the circle plate 111 is pushed and pulled, and as shown in FIG. 4 the camera 140 installed on the front of the refraction stacked material 110 is moved to an arrow direction (second direction). And, the refraction stacked material 110 may be formed in a straight line by the elasticity of the elastic supporter 130, when the pushed or pulled driving wire 120 is released from the adjustment part, simultaneously with retuning to an initial state in a camera 140.

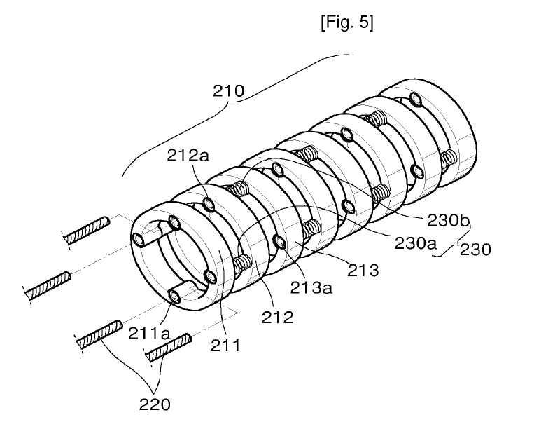

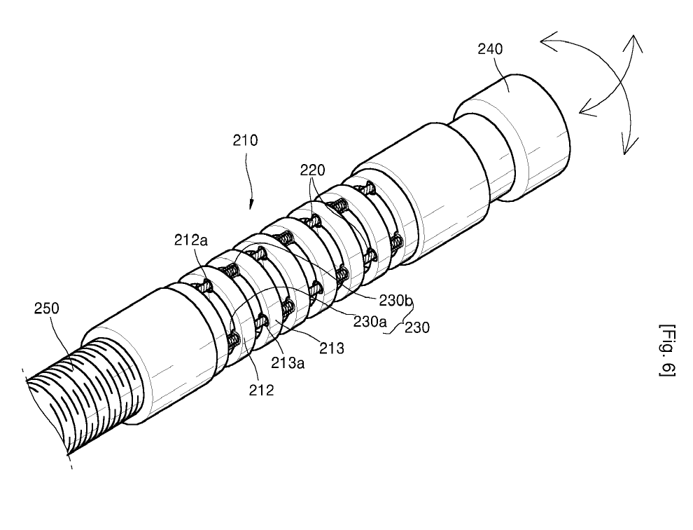

FIG. 5 is a disassembled perspective view showing third embodiment of a camera imaging apparatus with an endoscope according to the present invention, and FIG. 6 is a view showing a state at which a camera is able to be moved to fourth direction by a refraction stacked material in the camera imaging apparatus in FIG. 5.

As shown in FIGS. 5 and 6, third embodiment of the camera imaging apparatus with an endoscope according to the present invention includes a refraction stacked material 210 which is stacked to make a plenty of circle plates 211 to be refracted, a driving wire 220 which penetrates the refraction stacked material 210, an elastic supporter 230 which makes the refraction stacked material 210 to be able to be refracted, a camera 240 which is installed on the front of the refraction stacked material 210, and a hose 250 which is installed on the rear of the refraction stacked material 210. At this time, an adjustment part (not shown) at which an adjustor is able to make the manipulation of the camera imaging apparatus having an endoscope is installed on the rear of the hose 250. Further, the camera 240 is a known digital camera to be photographed by an image sensing device in a method of a charge-coupled device.

To explain the refraction stacked material 210, a circle plate represents first to third circle plates 211, 212, and 213 and the elastic supporter 230 represents first and second elastic supporters 230a and 230b.

Each first to third circle plates 211, 212, and 213 has four penetration holes 211a, 212a, and 213a formed with a predetermined separation of 90° on the center.

Moreover, first to third circle plates 211, 212, and 213 is respectively stacked so that a pair of first elastic supporter 230a is installed between penetration holes 211a and 212a formed oppositely against first and second circle plates 211 and 212 and a pair of second elastic supporter 230b is installed between penetration holes 212a and 213a positioned perpendicularly to first elastic supporter 230a formed between first and second circle plates 212 and 213. And, first and second circle plates and second and third circle plates is alternatively stacked. At this time, a driving wire 220 is formed so that the driving wire 220 may penetrate penetration holes of first to third circle plates 211, 212, and 213 and first and second elastic support 230a and 230b, respectively.

At the same time, the end part of the driving wire 220 is fixed to the upper of the refraction stacked material 210 and the lower thereof is coupled to the adjustment part via the hose 250.

The elastic supporter 230a and 230b makes the application of an elasticity force so that a plenty of first to third stacked circle plates 211 to 213 may be formed in a straight line. It is preferable that such elastic supporters 230a and 230b are made of a coil spring.

In such structure, the driving wire 220 which penetrate first to third penetration holes 211a, 212a, and 213a of first to third circle plates are pushed and pulled, and as shown in FIG. 6, a camera installed on the front of the refraction stacked material 210 is moved to an arrow direction (fourth direction). And, the refraction stacked material 210 may be formed in a straight line by the elasticity of the elastic supporter 230, when the pushed or pulled driving wire 220 is released from the adjustment part, simultaneously with retuning to an initial state in a camera 240.

Preferred embodiments of a camera imaging apparatus with an endoscope according to the present invention is an exemplary embodiment for reference, and therefore it is understandable that it is possible to make variable modifications and to carry out other equal embodiments from a skilled person who is known in the field of the present invention.

### INDUSTRIAL APPLICABILITY

According to a camera imaging apparatus with an endoscope of the present invention, an elastic supporter is supported so that the refraction stacked material may be made to be refracted and a wire makes the refraction stacked material refracted. Therefore, a camera is able to be moved first, second, or fourth direction even at a narrow space. In such camera imaging apparatus with an endoscope, a same circle plates are stacked by the elastic supporter, simultaneously with being penetrated by a driving wire, and thus it is possible to have a simple structure and it is almost little occurred to make out of order. Also, it is economic to have a manufacturing cost.

1. A camera imaging apparatus with an endoscope includes a refraction stacked material which is stacked to make a plenty of circle plate to be refracted, a driving wire which penetrates the circle plate of refraction stacked material, an elastic supporter which makes the circle plate of refraction stacked material to be able to be refracted, a camera which is installed on the front of the refraction stacked material, and a hose which is installed on the rear of the refraction stacked material and which penetrates the above driving wire; and
a refraction stacked material 110 having a plenty of circle plates 111 including a supporter 111a which is formed protuberantly less than a half circle to the upper of one side, a pair of supporting grooves 111b and 111c which is formed oppositely against across the outside of the supporter 111a, and a pair of penetration holes 111d and 111e which is formed oppositely against between the supporting grooves 111b and 111c, wherein the refraction stacked material 110 is realized in carrying out processes of making many circle plates 111 to be stacked to make the supporter 111a to be positioned at one part, and of inserting an elastic supporter 130 in the supporting grooves 111b and 111c and installing thereon, successively to make the penetration of a driving wire 120 through the penetration holes 111d and 111e.

2. A camera imaging apparatus with an endoscope according to claim 1, wherein the elastic supporter 130 is formed in that a concave groove 130a which is inserted in the border of the supporting grooves 111b and 111c has a constant distance so that the circle plates 111 may be keep a maintenance with a constant separation.

3. A camera imaging apparatus with an endoscope includes a refraction stacked material which is stacked to make a circle plate to be refracted, a driving wire which penetrates the circle plate of refraction stacked material, an elastic supporter which makes the circle plate of refraction stacked material to be able to be refracted, a camera which is installed on the front of the refraction stacked material, and a hose which is installed on the rear of the refraction stacked material and which penetrates the above driving wire;
a refraction stacked material 210 having first to third circle plates 211, 212, and 213 has four penetration holes 211a, 212a, and 213a formed with a pre-determined separation of 90° on the center;
an elastic supporter 230 representing first and second elastic supporters 230a and 230b which are respectively made of a coil spring;
first to third circle plates 211, 212, and 213 being respectively stacked so that a pair of first elastic supporter 230a is installed between penetration holes 211a and 212a formed oppositely against first and second circle plates 211 and 212 and a pair of second elastic supporter 230b is installed between penetration holes 212a and 213a positioned perpendicularly to first elastic supporter 230a formed between second and third circle plates 212 and 213; and
the driving wire 220 penetrating corresponding penetration holes 211a, 212a, and 213a of first to third circle plates 211, 212, and 213 and first and second elastic supporter 230a and 230b.
